(19) United States
(12) Patent Application Publication
(10) Pub. No.: US 2025/0013261 A1
(43) Pub. Date: Jan. 9, 2025

(54) Wrist-Worn Device with a Plurality of Displays Having a First Non-Coplanar Configuration and a Second Coplanar Configuration

(71) Applicant: Robert A. Connor, Wyoming, MN (US)

(72) Inventor: Robert A. Connor, Wyoming, MN (US)

(73) Assignee: Medibotics LLC, Ham Lake, MN (US)

(21) Appl. No.: 18/888,099

(22) Filed: Sep. 17, 2024

Related U.S. Application Data
(63) Continuation-in-part of application No. 18/226,213, filed on Jul. 25, 2023.
Application No. 18/226,213 is a continuation-in-part of application No. 17/871,952, filed on Jul. 24, 2022, now Pat. No. 11,747,769.
Application No. 17/871,952 is a continuation-in-part of application No. 17/515,509, filed on Oct. 31, 2021, now abandoned.
Application No. 17/515,509 is a continuation-in-part of application No. 16/926,748, filed on Jul. 12, 2020, now abandoned.
Application No. 16/926,748 is a continuation-in-part of application No. 16/819,147, filed on Mar. 15, 2020, now Pat. No. 11,429,151.
Application No. 17/515,509 is a continuation-in-part of application No. 16/926,748, filed on Jul. 12, 2020, now abandoned.
Application No. 16/819,147 is a continuation-in-part of application No. 16/598,514, filed on Oct. 10, 2019, now abandoned.
Application No. 16/598,514 is a continuation-in-part of application No. 14/623,337, filed on Feb. 16, 2015, now Pat. No. 9,582,035.
(60) Provisional application No. 62/876,213, filed on Jul. 19, 2019.
(60) Provisional application No. 62/882,560, filed on Aug. 4, 2019.
(60) Provisional application No. 62/820,337, filed on Mar. 19, 2019.
(60) Provisional application No. 62/115,691, filed on Feb. 13, 2015.
(60) Provisional application No. 62/113,423, filed on Feb. 7, 2015.
(60) Provisional application No. 62/111,163, filed on Feb. 3, 2015.
(60) Provisional application No. 62/106,856, filed on Jan. 23, 2015.
(60) Provisional application No. 62/100,217, filed on Jan. 6, 2015.
(60) Provisional application No. 61/948,124, filed on Mar. 5, 2014.
(60) Provisional application No. 61/944,090, filed on Feb. 25, 2014.

(51) Int. Cl. G06F 1/16

(52) U.S. Cl. CPC G06F 1/162; G06F 1/1624; G06F 1/163; G06F 1/1649; G06F 1/165

(57) ABSTRACT

A wrist-worn device such as a smart watch or band has a plurality of displays with a first non-coplanar configuration and a second coplanar configuration. When a larger display area is not needed, then the device is changed into the more-compact first configuration. When a larger display area is needed, then the device is changed into the larger-size second configuration.

### CROSS-REFERENCE TO RELATED APPLICATIONS

This application is a continuation in part of U.S. patent application Ser. No. 18/226,213 filed on 2023 Jul. 25. U.S. patent application Ser. No. 18/226,213 was a continuation in part of U.S. patent application Ser. No. 17/871,952 filed on 2022 Jul. 24. U.S. patent application Ser. No. 17/871,952 was a continuation in part of U.S. patent application Ser. No. 17/515,509 filed on 2021 Oct. 31. U.S. patent application Ser. No. 17/871,952 was a continuation in part of U.S. patent application Ser. No. 16/926,748 filed on 2020 Jul. 12. U.S. patent application Ser. No. 17/871,952 was a continuation in part of U.S. patent application Ser. No. 16/819,147 filed on 2020 Mar. 15.

U.S. patent application Ser. No. 17/515,509 was a continuation in part of U.S. patent application Ser. No. 16/926,748 filed on 2020 Jul. 12. U.S. patent application Ser. No. 16/926,748 was a continuation in part of U.S. patent application Ser. No. 15/431,769 filed on 2017 Feb. 14. U.S. patent application Ser. No. 16/926,748 claimed the priority benefit of U.S. provisional patent application 62/876,213 filed on 2019 Jul. 19. U.S. patent application Ser. No. 16/819,147 was a continuation in part of U.S. patent application Ser. No. 16/598,514 filed on 2019 Oct. 10. U.S. patent application Ser. No. 16/819,147 was a continuation in part of U.S. patent application Ser. No. 15/294,746 filed on 2016 Oct. 16 which issued as U.S. Pat. No. 10,627,861 on 2020 Apr. 21. U.S. patent application Ser. No. 16/819,147 claimed the priority benefit of U.S. provisional patent application 62/882,560 filed on 2019 Aug. 4. U.S. patent application Ser. No. 16/819,147 claimed the priority benefit of U.S. provisional patent application 62/820,337 filed on 2019 Mar. 19.

U.S. patent application Ser. No. 16/598,514 was a continuation in part of U.S. patent application Ser. No. 15/294,746 filed on 2016 Oct. 16 which issued as U.S. Pat. No. 10,627,861 on 2020 Apr. 21. U.S. patent application Ser. No. 16/598,514 was a continuation in part of U.S. patent application Ser. No. 14/623,337 filed on 2015 Feb. 16 which issued as U.S. Pat. No. 9,582,035 on 2017 Feb. 28. U.S. patent application Ser. No. 15/431,769 was a continuation in part of U.S. patent application Ser. No. 14/623,337 filed on 2015 Feb. 16 which issued as U.S. Pat. No. 9,582,035 on 2017 Feb. 28. U.S. patent application Ser. No. 15/294,746 was a continuation in part of U.S. patent application Ser. No. 14/623,337 filed on 2015 Feb. 16 which issued as U.S. Pat. No. 9,582,035 on 2017 Feb. 28.

U.S. patent application Ser. No. 14/623,337 claimed the priority benefit of U.S. provisional patent application 62/115,691 filed on 2015 Feb. 13. U.S. patent application Ser. No. 14/623,337 claimed the priority benefit of U.S. provisional patent application 62/113,423 filed on 2015 Feb. 7. U.S. patent application Ser. No. 14/623,337 claimed the priority benefit of U.S. provisional patent application 62/111,163 filed on 2015 Feb. 3. U.S. patent application Ser. No. 14/623,337 claimed the priority benefit of U.S. provisional patent application 62/106,856 filed on 2015 Jan. 23. U.S. patent application Ser. No. 14/623,337 claimed the priority benefit of U.S. provisional patent application 62/100,217 filed on 2015 Jan. 6. U.S. patent application Ser. No. 14/623,337 claimed the priority benefit of U.S. provisional patent application 61/948,124 filed on 2014 Mar. 5. U.S. patent application Ser. No. 14/623,337 claimed the priority benefit of U.S. provisional patent application 61/944,090 filed on 2014 Feb. 25.

The entire contents of these applications are incorporated herein by reference.

### FEDERALLY SPONSORED RESEARCH

Not Applicable

### SEQUENCE LISTING OR PROGRAM

Not Applicable

### BACKGROUND

### Field of Invention

This invention relates to wrist-worn electronic devices with displays.

### Introduction

Smart watches have several advantages over handheld mobile devices. For example, smart watches can incorporate biometric sensors and are less cumbersome than a cell phone for athletic activities. However, a disadvantage of current smart watches is their small screen size.

### Review of the Relevant Art

U.S. Pat. No. 6,035,035 (Firooz, Mar. 7, 2000, “Wrist Mounted Telephone Device”) discloses a wrist-mounted telephone. U.S. patent application publication 20020021622 (Baroche, Feb. 21, 2002, “Multifunction Wristwatch with Electronic Device and Foldable Display Screen”) discloses a wrist watch with an unfolding display. U.S. Pat. No. 6,970,157 (Siddeeq, Nov. 29, 2005, “Wearable Computing, Input, and Display Device”) discloses a wearable device with a display which can be rotated from a flat position to a raised position.

U.S. Pat. No. 7,558,057 (Naksen et al., Jul. 7, 2009, “Personal Digital Device with Adjustable Interface”) discloses a personal digital device with a screen with variable stiffness and size. U.S. patent application publication 20090219788 (Henley, Sep. 3, 2009, “Combination Watch and Cell Phone Foldable onto Each Other for Use Around a Wrist of a User”) discloses a combination watch and phone which fold onto each other. U.S. patent application publication 20110187681 (Kim et al., Aug. 4, 2011, “Apparatus for Screen Location Control of Flexible Display”) discloses an apparatus to control an image output position of a flexible display.

U.S. patent application publication 20130044215 (Rothkopf, Feb. 21, 2013, “Bi-Stable Spring with Flexible Display”) discloses a wearable accessory device with a flexible display coupled to a bi-stable spring. U.S. Pat. No. 8,851,372 (Zhou et al., Oct. 7, 2014, “Wearable Personal Digital Device with Changeable Bendable Battery and Expandable Display Used As Standalone Electronic Payment Card”) discloses a wearable device and methods for enlarging its display. U.S. patent application publication 20150029227 (Park et al., Jan. 29, 2015, “Wrist-Wearable Display Apparatus and Method for Controlling the Same”) discloses a wrist-wearable apparatus with a hidden display which is exposed if a main display is rotated.

U.S. patent application publication 20150113473 (Otsuka et al., Apr. 23, 2015, “Electronic Device”) discloses a wearable touch panel with a first display region and a second display region contiguous with and narrower than the first display region. U.S. patent application publication 20150338882 (Yun et al., Nov. 26, 2015, “Electronic Device with Foldable Display and Method of Operating the Same”) discloses a method of operating an electronic device having a foldable display.

U.S. patent application publication 20160239190 (Forutanpour et al., Aug. 18, 2016, “Efficient Display of Content on Wearable Displays”) discloses systems, methods, and devices for displaying information in various display regions of a wearable display device, wherein a processor may re-size an image and/or deactivate a portion of the display. U.S. patent application publication 20160240154 (Forutanpour et al., Aug. 18, 2016, “Efficient Operation of Wearable Displays”) discloses systems, methods, and devices for displaying information in various display regions of a wearable display device in order to extend battery life.

U.S. patent application publication 20160246558 (Prushinskiy et al., Aug. 25, 2016, “Foldable Display”) discloses a foldable display panel including first to fourth regions adjacent to each other. U.S. patent application publication 20160267310 (Al Nasser et al., Sep. 15, 2016, “Wearable Device”) discloses a wearable device with a first wrist band unit extended in an opposite direction from a second wrist band unit. U.S. Pat. No. 9,848,494 (Huitema et al., Dec. 19, 2017, “Support Structures for a Flexible Electronic Component”) discloses a dynamically flexible article or device, such as a wristband, an armband, a rollable e-reader, or a belt, which includes a flexible display. U.S. Pat. No. 9,877,384 (Lee et al., Jan. 23, 2018, “Display Device”) discloses a display device with a flexible display panel and a housing having an outer peripheral surface around which the flexible display panel is rolled. U.S. patent application publication 20180032106 (Yu et al., Feb. 1, 2018, “Rollable Flexible Display Device”) discloses a flexible display that rolls or unrolls like a scroll.

U.S. patent application publication 20180120901 (Jin et al., May 3, 2018, “Foldable Display Device and Electronic Apparatus with the Same and Control Method of the Same”) and U.S. Pat. No. 10,216,227 (Jin et al., Feb. 26, 2019, “Foldable Display Device and Electronic Apparatus with the Same and Control Method of the Same”) disclose a foldable display device with a first display screen, a second display screen, a third display screen, a first connector connecting the first and second screens, and a second connector connecting the second and third screens, wherein the second screen can rotate around the first connector and the third screen can rotate around the second connector.

U.S. patent application publication 20180137801 (An, May 17, 2018, “Flexible Display Device and Displaying Method of Flexible Display Device”) discloses a flexible display device including a flexible display and a sensor which obtains user gaze information. U.S. Pat. No. 10,152,028 (Kim, Dec. 11, 2018, “Wristwatch Type Smart Terminal”) discloses a wristwatch type smart terminal with a display which can slide or rotate on a band. U.S. Pat. No. 10,299,391 (Kim et al., May 21, 2019, “Rollable Display Device”) discloses a rollable display device with a flexible display, including a main roller around which the flexible display is wound and a link drive unit for moving the flexible display.

U.S. Pat. No. 10,319,263 (Lee et al., Jun. 11, 2019, “Flexible Display”) discloses a flexible display with a fixing part which has a restoring force that presses on the upper surface of the flexible display. U.S. Pat. No. 10,319,331 (Pasupathi, Jun. 11, 2019, “Variable Display Size for an Electronic Display Device”) and U.S. Pat. No. 10,755,668 (Pasupathi, Aug. 25, 2020, “Variable Display Size for an Electronic Display Device”) disclose an electronic display device configured for variable display size. U.S. patent application publication 20190196550 (Kim et al., Jun. 27, 2019, “Flexible Display Window and Electronic Device Having the Same”) discloses a display device with a first portion of a screen which is exposed through a rigid portion and a second of the screen which is extended from the first portion and exposed through a flexible portion.

U.S. patent application publication 20190229288 (Ahn et al., Jul. 25, 2019, “Foldable Display Apparatus”) discloses a display that is adjustable between a folded configuration and an unfolded configuration. U.S. patent application publication 20190237532 (Ahn et al., Aug. 1, 2019, “Foldable Display Device”) discloses a display panel including a bending portion, and a folding unit supporting the display panel, wherein the display device is capable of being in-folded and out-folded. U.S. patent application publication 20190268455 (Back et al., Aug. 29, 2019, “Electronic Device Including Movable Flexible Display”) discloses a device with an assembly that enables varying a visible portion of a display by extending and retracting a bendable portion of the display.

U.S. patent application publication 20190268771 (Seo et al., Aug. 29, 2019, “Mobile Device of Bangle Type, Control Method Thereof, and UI Display Method”) discloses a bangle-type device with a display screen which is changed based on motion. U.S. patent application publication 20190278336 (Choi et al., Sep. 12, 2019, “Electronic Device with Flexible Display and Method for Operating Same”) discloses a device with a slidable flexible display. U.S. patent application publication 20190296259 (Baek et al., Sep. 26, 2019, “Electronic Device and Method of Forming Flexible Display Thereof”) discloses an electronic device with a display and flexible layer disposed in a recess.

U.S. patent application publication 20190305237 (Shin et al., Oct. 3, 2019, “Electronic Device With Movable Flexible Display and Operating Method Thereof”) discloses a flexible touchscreen layer movable between an open state and a closed state. U.S. Pat. No. 10,517,180 (Choi et al., Dec. 24, 2019, “Display Device and Method for Driving the Same”) discloses a display device with a flexible display and a rolling driver. U.S. patent application publication 20200042037 (Sun, Feb. 6, 2020, “Wearable Display Device”) discloses a wearable display device with a flexible screen and a support component supporting the flexible screen.

U.S. Pat. No. 10,617,017 (Park et al., Apr. 7, 2020, “Rollable Display”) discloses a rollable display that can be rolled up and down. U.S. Pat. No. 10,802,544 (Kwak et al., Oct. 13, 2020, “Flexible Display Device and Method of Controlling Same”) discloses a flexible display device with a sensor configured to detect at least one rolling characteristic in response to the display being rolled. U.S. Pat. No. 10,820,433 (Cha, Oct. 27, 2020, “Flexible Display Device”) discloses a flexible display device with a folded state. U.S. Pat. No. 10,827,630 (Kim et al., Nov. 3, 2020, “Rollable Display”) discloses a roller that is bonded to one end of a flexible display panel and allows the flexible display panel to be rolled. U.S. patent Ser. No. 10/877,525 (Kang et al., Dec. 29, 2020, “Rollable Display”) discloses a rollable display, wherein a flexible display panel is rolled onto a panel guide.

U.S. Pat. No. 10,937,393 (Lee et al., Mar. 2, 2021, “Electronic Device Including Flexible Display and Content Display Method Thereof”) discloses a flexible display that is rolled up and a sensor configured to detect unrolling of the flexible display. U.S. Pat. No. 10,969,830 (In et al., Apr. 6, 2021, “Flexible Display Device and Method for Compensating Image of Flexible Display Device”) discloses a rollable display panel and an optical sensor to detect luminance of the rollable display panel. U.S. patent application publication 20210104582 (Kim et al., Apr. 8, 2021, “Display Apparatus and Electric Apparatus Including the Same”) discloses a flexible display panel displaying an image via an upper surface.

U.S. patent application publication 20210143333 (Sugitani et al., May 13, 2021, “Display Apparatus and Method of Manufacturing the Same”) discloses a display with a substrate, a conductive layer on the substrate, and an insulating pattern on the conductive layer. U.S. Pat. No. 11,016,532 (Yang, May 25, 2021, “Display Device and Moving Method Thereof”) discloses a display device with a first roller, a second roller, and a gear coupled to the second roller. U.S. patent application publication 20210173487 (Ham, Jun. 10, 2021, “Display Apparatus”) discloses a display panel and a vibration generating device which vibrates the display panel.

U.S. patent application publication 20210182008 (Kim, Jun. 17, 2021, “Display Device”) discloses a display device with a roller and a rollable display whose length is adjusted by rotation of the roller. U.S. patent application publication 20210192989 (Ahn et al., Jun. 24, 2021, “Display Device and Method of Providing the Same”) discloses a display device with a flexible substrate. U.S. patent application publication 20210201713 (Chung et al., Jul. 1, 2021, “Display Apparatus”) discloses a display configured to be wound around first and second rolls.

U.S. patent application publication 20210208636 (Kim et al., Jul. 8, 2021, “Display Device”) discloses a display panel with a protective layer. U.S. patent application publication 20210217839 (Choi et al., Jul. 15, 2021, “Display Apparatus and Method of Manufacturing the Same”) discloses a display apparatus with a thin film transistor. U.S. patent application publication 20210216108 (Lee et al., Jul. 15, 2021, “Flexible Display Device”) discloses a flexible display that can change an area exposed to the outside. U.S. patent application publication 20210226181 (Seo et al., Jul. 22, 2021, “Display Apparatus and Method of Manufacturing the Same”) discloses a method of manufacturing a display including a thin film encapsulation layer on a surface of a flexible substrate.

U.S. patent application publication 20210303121 (Ku, Sep. 30, 2021, “Display Device”) discloses a base member configured to be folded or unfolded along a folding axis and including a display area. U.S. patent application publication 20210318730 (Lee et al., Oct. 14, 2021, “Display Device”) discloses a display device with a substrate, a first sensor electrode, and a second sensor electrode disposed on the substrate. U.S. patent application publication 20210320163 (Bang et al., Oct. 14, 2021, “Display Device”) discloses a base with a display area including a plurality of pixels each having at least one switching element and a light-emitting element.

U.S. patent application publication 20210367020 (Bok et al., Nov. 25, 2021, “Foldable Display Device, Rollable Display Device, and Display Device”) discloses a foldable display device including a front surface and a rear surface opposite the front surface, wherein the front surface includes a first area with a first transmissive portion and a second area with a second transmissive portion. U.S. patent application publication 20210376033 (Chae et al., Dec. 2, 2021, “Display Panel and Electronic Apparatus”) discloses a display panel with a substrate, two pixels on the substrate, and a blocking layer.

U.S. patent application publication 20210384179 (Bok et al., Dec. 9, 2021, “Display Device”) discloses a first display area including first sub-pixels to display an image and a second display area including second sub-pixels. U.S. patent application publication 20220052121 (Jang et al., Feb. 17, 2022, “Display Device”) discloses a display device including a first base portion and a second base portion. U.S. patent application publication 20220139326 (Bae et al., May 5, 2022, “Display Device”) discloses a display panel which displays an image during a plurality of driving frames.

U.S. patent application publication 20220149322 (Kim et al., May 12, 2022, “Display Apparatus and Method of Manufacturing the Same”) discloses a display apparatus with a substrate including a polymer resin and a protective layer. U.S. patent application publication 20220155825 (Kim et al., May 19, 2022, “Display Device”) discloses a display panel including an area of a constant shape and a shock absorber. U.S. patent application publication 20220157250 (Kim et al., May 19, 2022, “Scan Driver and Display Device Having the Same”) discloses a scan driver of a display device including a driving circuit and a masking circuit.

U.S. patent application publication 20220187874 (Chun et al., Jun. 16, 2022, “Rollable Mobile Terminal”) discloses a rollable mobile terminal with a flexible display unit and frame. U.S. patent application publication 20220291718 (Park, Sep. 15, 2022, “Flexible Display Device and Control Method Therefor”) discloses a flexible display device and a control method therefor. U.S. patent application publication 20220317732 (Lee et al., Oct. 6, 2022, “Flexible Display Apparatus”) discloses a flexible display apparatus with a rollable display and a polygonal-prism-shaped roller.

U.S. Pat. No. 11,527,179 (Han et al., Dec. 13, 2022, “Display Device”) discloses a display panel and a roller around which the display panel is wound or unwound. U.S. Pat. No. 11,576,270 (Rha et al., Feb. 7, 2023, “Display Apparatus”) discloses a housing provided with an opening at a front surface, a roller disposed within the housing, and a display which is wound around the roller. U.S. Pat. No. 11,592,874 (Seger et al, Feb. 28, 2023, “Touch Sensing in a Flexible/Foldable Touch Screen Display”) discloses a device having a flexible touch screen display configured to display images in at least a first touch area and a second touch area.

U.S. patent application publication 20230076158 (Li et al., Mar. 9, 2023, “Self-Adaptive Display Aspect Ratio Adjuster and Gesture on Rolling Devices”) discloses a device and method for optimizing the size of visual content on a rollable display device. U.S. Pat. No. 11,614,777 (Kishimoto et al., Mar. 28, 2023, “Electronic Apparatus and Method for Manufacturing the Same”) discloses a display panel having non-folding areas and a folding area between the non-folding areas. U.S. patent application publication 20230132649 (Park, May 4, 2023, “Display Device and Method of Manufacturing the Same”) discloses a display device with a substrate having a bent portion and a bending protective layer.

U.S. Pat. No. 11,647,598 (Zhang et al., May 9, 2023, “Display Apparatus”) discloses a device with a flexible display screen, a fixator, a rotation rod, and an armrest. U.S. patent application publication 20230169901 (Wu et al., Jun. 1, 2023, “Display Panel and Display Device”) discloses a rollable display panel with a display region and a non-display region surrounding the display region. U.S. Pat. No. 11,699,372 (Lee et al., Jul. 11, 2023, “Electronic Device Including a Flexible Display with Variable Region and Method of Heating the Variable Region of the Flexible Display”) discloses an operating method of an electronic device that includes measuring an external temperature via a sensor.

### SUMMARY OF THE INVENTION

Disclosed herein is a wrist-worn device such as a smart watch or band with two or more displays which have a first non-coplanar configuration and a second coplanar configuration. This device helps to address the small-screen limitation of wrist-worn devices such as smart watches. When a larger display area is not needed, then the device can be changed into the more-compact first configuration. When a larger display area is needed, then the device can be changed into the larger-size second configuration.

In an example, the device can be changed between a first configuration to a second configuration by rotating and/or pivoting a second display relative to a first display. In an example, the device can be changed between a first configuration to a second configuration by sliding and/or telescoping a second display relative to a first display. In an example, the device can be changed between a first configuration to a second configuration by flipping and/or unfolding a second display relative to a first display.

### BRIEF INTRODUCTION TO THE FIGURES

FIGS. 1 and 2 show two dorsal views, at two different times, of a wearable device with a plurality of displays having a first non-coplanar configuration and a second coplanar configuration, wherein there is one display visible in the first non-coplanar configuration and there are two displays visible in the second coplanar configuration.

FIG. 1 shows this device at a first time in the first non-coplanar configuration.

FIG. 2 shows this device at a second time in the second coplanar configuration.

FIGS. 3 and 4 show two dorsal views, at two different times, of a wearable device with a plurality of displays having a first non-coplanar configuration and a second coplanar configuration, wherein there is one display visible in the first non-coplanar configuration and there are three displays visible in the second coplanar configuration.

FIG. 3 shows this device at a first time in the first non-coplanar configuration.

FIG. 4 shows this device at a second time in the second coplanar configuration.

### DETAILED DESCRIPTION OF THE FIGURES

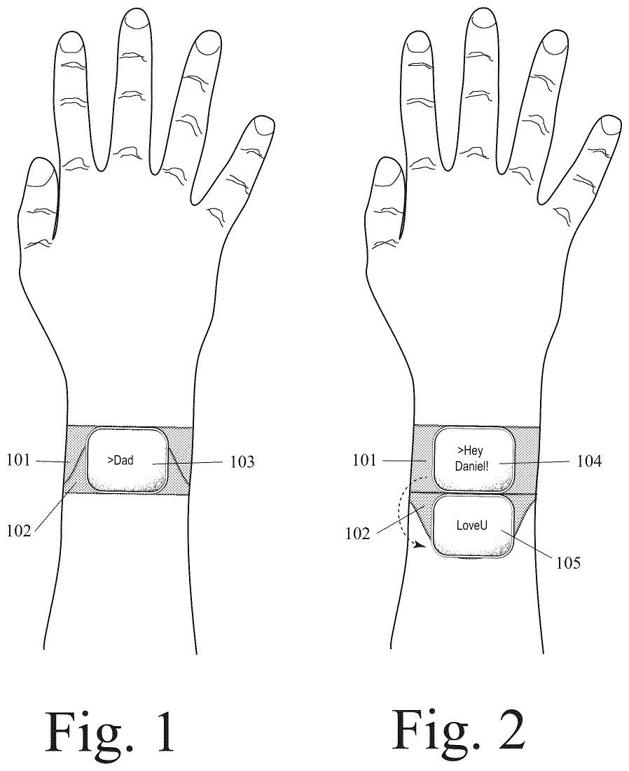

Before discussing the specific embodiments of this invention which are shown in FIGS. 1 through 4, this disclosure provides an introductory section which covers the general concepts, components, and methods which comprise this invention. Where relevant, these concepts, components, and methods can be applied as variations to the examples shown in FIGS. 1 through 4 which are discussed afterwards.

In an example, a wearable computing device can comprise: a primary attachment member, wherein this primary attachment member is configured to attach the device to a person's wrist and/or forearm by encircling at least 50% of the circumference of the person's wrist and/or forearm; a folding attachment member, wherein this folding attachment member has a first configuration in which it overlaps the primary attachment member by a first amount, wherein this folding attachment member has a second configuration in which it overlaps primary attachment member by a second amount, wherein the second amount is less than the first amount, and wherein the folding attachment member is moved from the first configuration to the second configuration by rotation of the folding attachment member relative to the primary attachment member; a first display member, wherein this first display member is on the exterior surface of the device and visible to the person when the folding attachment member is in the first configuration; and a second display member, wherein this second display member is on the exterior surface of the device and visible to the person when the folding attachment member is in the second configuration.

In an example, the device can further comprise a third display member, wherein this third display member is on the exterior surface of the device and visible to the person when the folding attachment member is in the second configuration. In an example, the primary attachment member can be a band, strap, chain, bracelet, or bangle. In an example, the folding attachment member can have arcuate edges which connect to the lateral sides of the second display member. In an example, at least 90% of the folding attachment member can overlap the primary attachment member in the first configuration. In an example, less than 10% of the folding attachment member can overlap primary attachment member in the second configuration. In an example, a display member can be a computer display screen.

In an example, a wearable computing device can comprise: a primary attachment member, wherein this primary attachment member is configured to attach the device to a person's wrist and/or forearm by encircling at least 50% of the circumference of the person's wrist and/or forearm; a folding attachment member, wherein this folding attachment member has a first configuration in which it overlaps the primary attachment member by a first amount, wherein this folding attachment member has a second configuration in which it overlaps primary attachment member by a second amount, wherein the second amount is less than the first amount, and wherein the folding attachment member is moved from the first configuration to the second configuration by rotation of the folding attachment member relative to the primary attachment member; a first display member, wherein this first display member is on the exterior surface of the device and visible to the person when the folding attachment member is in the first configuration; a second display member, wherein this second display member is on the exterior surface of the device and visible to the person when the folding attachment member is in the second configuration; and; a third display member, wherein this third display member is on the exterior surface of the device and visible to the person when the folding attachment member is in the second configuration.

In an example, the primary attachment member can be a band, strap, chain, bracelet, or bangle. In an example, the folding attachment member can have arcuate edges which connect to the lateral sides of the second display member. In an example, at least 90% of the folding attachment member can overlap the primary attachment member in the first configuration. In an example, less than 10% of the folding attachment member can overlap the primary attachment member in the second configuration. In an example, a display member can be a computer display screen.

In an example, a wearable computing device can comprise: a first display which is attached to a person's wrist and/or forearm; and a second display which is attached to the person's wrist and/or forearm, wherein the first display and the second display have a first configuration in which the first display and the second display are not coplanar, wherein the first display and the second display have a second configuration in which the first display and the second display are coplanar.

In an example, the device can further comprise a band or strap which attaches the first display and the second display to the person's wrist and/or forearm. In an example, the first display and the second display can be moved from their first configuration to their second configuration by rotating and/or pivoting the second display relative to the first display. In an example, the first display and the second display can be moved from their first configuration to their second configuration by sliding and/or telescoping the second display relative to the first display. In an example, the first display and the second display can be moved from their first configuration to their second configuration by flipping and/or unfolding the second display relative to the first display.

In an example, the first display can be on the dorsal quadrant of the person's wrist and/or forearm, the second display can be on the ventral quadrant of the person's wrist and/or forearm in the first configuration, and the second display can be on the dorsal quadrant of the person's wrist and/or forearm in the second configuration. In an example, the first display can be on the dorsal quadrant of the person's wrist and/or forearm, the second display can be between the dorsal and ventral quadrants of the person's wrist and/or forearm in the first configuration, and the second display can be on the dorsal quadrant of the person's wrist and/or forearm in the second configuration.

In an example, a wearable device can comprise: a plurality of (computer and/or digital) displays which are attached to a person's wrist and/or forearm, wherein the plurality of displays have a first configuration in which they are not coplanar, wherein the plurality of displays have a second configuration in which they are coplanar, and wherein the plurality of displays are moved from their first (non-coplanar) configuration to their second (coplanar) configuration by a mechanism selected from the group consisting of: rotating and/or pivoting a second display relative to a first display, sliding and/or telescoping a second display relative to a first display, and flipping and/or unfolding a second display relative to a first display.

In an example, a wearable device can comprise: a first (computer and/or digital) display which is attached to a person's wrist and/or forearm; and a second (computer and/or digital) display which is attached to the person's wrist and/or forearm, wherein the first display and the second display have a first configuration in which the first display and the second display are not coplanar, wherein the first display and the second display have a second configuration in which the first display and the second display are coplanar, and wherein the first display and the second display are moved from their first (non-coplanar) configuration to their second (coplanar) configuration by a mechanism selected from the group consisting of: rotating and/or pivoting the second display relative to the first display, sliding and/or telescoping the second display relative to the first display, and flipping and/or unfolding the second display relative to the first display.

In an example, the wearable device can further comprise a flexible strip around which the second display rotates and/or pivots between the first (non-coplanar) configuration and the second (coplanar) configuration. In an example, a first display can be on the dorsal side (e.g. dorsal quadrant) of a person's wrist and/or forearm, a second display can be on the ventral side (e.g. ventral quadrant) of the person's wrist and/or forearm in the first (non-coplanar) configuration, and the second display can be on the dorsal side (e.g. dorsal quadrant) of the person's wrist and/or forearm in the second (coplanar) configuration.

In an example, the center of a first display and the center of a second display can be separated by a first distance in their first (non-coplanar) configuration and by a second distance in their second (coplanar) configuration, wherein the second distance is less than the first distance. In an example, a device can further comprise band (or strap) which attaches the first and second displays to a person's wrist and/or forearm, wherein the band (or strap) is (proximally-vs-distally) asymmetric with respect to a circumferential line around the person's wrist and/or forearm, wherein a proximal portion of the band (or strap) which is distal to the circumferential line spans the entire circumference of the person's wrist and/or forearm and a distal portion of the band (or strap) which is proximal to the circumferential line does not span the entire circumference of the person's wrist and/or forearm. In an example, a first display can be dorsal (e.g. closer to the most dorsal point of a circumference of a person's wrist and/or forearm) relative to a second display in a first (non-coplanar) configuration.

In an example, in a first (non-coplanar) configuration, a first display can be located at a first radial and/or polar-coordinate location on the circumference of a person's wrist and/or forearm and a second display can be located at a second radial and/or polar-coordinate location on this circumference, wherein the first and second radial and/or polar-coordinate locations differ by between 10 and 30 degrees. In an example, in their first (non-coplanar) configuration, a first display can be in a first plane in 3D space and a second display can be in a second plane in 3D space, wherein the first plane and the second plane intersect each other in 3D space at an angle in the range of 20 to 40 degrees. In an example, the display surfaces of the first and second displays can be non-coplanar in their first configuration. In an example, a first display and a second display can be adjacent in the second (coplanar) configuration.

In an example, a device can further comprise band (or strap) which attaches the first and second displays to a person's wrist and/or forearm, wherein the band (or strap) is (proximally-vs-distally) asymmetric with respect to a circumferential line around the person's wrist and/or forearm. In an example, a first display and a second display can show two portions of the same (image) content in a second configuration. In an example, first and second planes can be both coplanar and aligned along the same proximal-to-dorsal vector in the second configuration. In an example, in a second configuration, the centroids of a first display and of a second display can be aligned along the same proximal-to-distal vector on a person's wrist and/or forearm, where proximal means closer to the person's elbow and distal means farther from the person's elbow.

In an example, the device can further comprise a band (or strap) which attaches the first display and the second display to a person's wrist and/or forearm, wherein the second display is connected to the band (or strap) by two half-parabolic, half-wave-phase, and/or half-sinusoidal-phase members (e.g. strips). In an example, a device can further comprise a hinge around which a second display rotates and/or pivots between a first (non-coplanar) configuration and a second (coplanar) configuration. In an example, a first display and a second display can be changed from their first (non-coplanar) configuration to their second (coplanar) configuration by moving only the first display. In an example, a second display can be dorsal (e.g. closer to the most dorsal point of a circumference of a person's wrist and/or forearm) relative to a first display.

In an example, in a second (coplanar) configuration, a first display can be located at a first radial and/or polar-coordinate location on the circumference of a person's wrist and/or forearm and a second display can be located at a second radial and/or polar-coordinate location on this circumference, wherein the first and second radial and/or polar-coordinate locations differ by between 60 and 100 degrees. In an example, the center of a first display and the center of a second display can be separated by a first distance in their first (non-coplanar) configuration and by a second distance in their second (coplanar) configuration, wherein the second distance is at least 50% greater than the first distance. In an example, a device can comprise three displays, wherein a one of the three displays is visible in a first configuration and two of the three displays are visible in a second configuration.

In an example, in a second (coplanar) configuration, a first display can be located at a first radial and/or polar-coordinate location on the circumference of a person's wrist and/or forearm and a second display can be located at a second radial and/or polar-coordinate location on this circumference, wherein the first and second radial and/or polar-coordinate locations differ by between 20 and 50 degrees.

In an example, a device can further comprise a band (or strap) around a person's wrist and/or forearm, wherein the band (or strap) is connected to opposite sides of a first display, and wherein the band (or strap) is connected to a second display by elastic and/or flexible components (e.g. strips). In an example, a first display can be proximal (e.g. closer to a person's elbow) relative to a second display in a second (coplanar) configuration. In an example, in a first configuration, a first display and a second display can be located on different circumferential lines around a person's wrist and/or forearm. In an example, in their first (non-coplanar) configuration, a first display can be in a first plane in 3D space and a second display can be in a second plane in 3D space, wherein the first plane and the second plane intersect each other in 3D space at a 60 degree angle. In an example, a device can further comprise a hinge around which a second display is manually rotated and/or pivoted between a first (non-coplanar) configuration and a second (coplanar) configuration.

In an example, a first display and a second display can be changed from their first (non-coplanar) configuration to their second (coplanar) configuration by moving both the first display and the second display. In an example, a second display can be dorsal (e.g. closer to the most dorsal point of a circumference of a person's wrist and/or forearm) relative to a first display in a first (non-coplanar) configuration. In an example, in a second configuration, a first display and a second display can both be located on the same circumferential line around a person's wrist and/or forearm. In an example, the device can further comprise a band (or strap) which attaches the first display and the second display to a person's wrist and/or forearm. In an example, a device can further comprise a joint around which a second display rotates and/or pivots between the first (non-coplanar) configuration and the second (coplanar) configuration.

In an example, a first display and a second display can be separated by a first distance in their first (non-coplanar) configuration and by a second distance in their second (coplanar) configuration, wherein the second distance is less than the first distance. In an example, a second display can be dorsal (e.g. closer to the most dorsal point of a circumference of a person's wrist and/or forearm) relative to a first display in a second (coplanar) configuration. In an example, in a second configuration, a first display and a second display can be located on different circumferential lines around a person's wrist and/or forearm. In an example, the device can further comprise a band (or strap) which attaches the first display and the second display to a person's wrist and/or forearm, wherein two sides of a first display are connected to the band (or strap).

In an example, a device can further comprise a joint around which a second display is manually rotated and/or pivoted between the first (non-coplanar) configuration and the second (coplanar) configuration. In an example, a first display and a second display can be separated by a first distance in their first (non-coplanar) configuration and by a second distance in their second (coplanar) configuration, wherein the second distance is greater than the first distance. In an example, a second display can be larger than a first display. In an example, in a second configuration, a first display is visible to a person but a second display is not visible to the person. In an example, the device can further comprise a band (or strap) which attaches the first display and the second display to a person's wrist and/or forearm, wherein two sides of a first display are connected to the band (or strap) and two sides of a second display are connected to the band (or strap).

In an example, a device can further comprise a band (or strap), wherein the band (or strap) is connected to between 50% and 80% of the perimeter of the second display. In an example, a display can be a watch face. In an example, a first display can show a first portion and a second display can show a second portion of the same (image) content in a second configuration, wherein the size (e.g. area) of second portion is between 40% and 60% of the size (e.g. area) of the first portion. In an example, in a first configuration, the centroids of a first display and of a second display can be aligned along the same proximal-to-distal vector on a person's wrist and/or forearm, where proximal means closer to the person's elbow and distal means farther from the person's elbow.

In an example, in their first (non-coplanar) configuration, first and second displays can be parallel. In an example, a device can further comprise a lock mechanism which reversibly locks a first display and/or a second display into their second configuration. In an example, a first display and a second display can be separated by a first distance in their first (non-coplanar) configuration and by a second distance in their second (coplanar) configuration, wherein the second distance is at least 50% greater than the first distance. In an example, a second display can be proximal (e.g. closer to a person's elbow) relative to a first display. In an example, in a second configuration, a second display can reversibly snap, clip, or latch into a coplanar location with respect to a first display.

In an example, the device can further comprise a band (or strap) which attaches the first display and the second display to a person's wrist and/or forearm, wherein two sides of a first display are connected to the band (or strap) and three sides of a second display are connected to the band (or strap). In an example, a device can further comprise a lock mechanism which reversibly locks a first display and/or a second display into their second configuration, wherein the lock mechanism is selected from the group consisting of: snap, clip, clasp, latch, prong, pin, spring, and magnet. In an example, a first display and a second display can be the same size. In an example, a second display can be proximal (e.g. closer to a person's elbow) relative to a first display in a first (non-coplanar) configuration.

In an example, in a second configuration, a second display is visible to a person but a first display is not visible to the person. In an example, the device can further comprise a band (or strap) which attaches the first display and the second display to a person's wrist and/or forearm, wherein three sides of a first display are connected to the band (or strap) and two sides of a second display are connected to the band (or strap). In an example, a device can further comprise an (electromagnetic) actuator which automatically moves a first display and/or a second display from their first (non-coplanar) configuration to the second (coplanar) configuration. In an example, a first display and a second display can display different (image) content.

In an example, a second display can be proximal (e.g. closer to a person's elbow) relative to a first display in a second (coplanar) configuration. In an example, in a second configuration, both a first display and a second display are visible to a person. In an example, the device can further comprise a band (or strap) which attaches the first display and the second display to a person's wrist and/or forearm, wherein the second display is connected to the band (or strap) by two arcuate (e.g. concave) strips. In an example, a device can further comprise band (or strap) which attaches the first and second displays to a person's wrist and/or forearm, wherein the band (or strap) is (proximally-vs-distally) asymmetric with respect to a circumferential line around the person's wrist and/or forearm, wherein a distal portion of the band (or strap) which is distal to the circumferential line spans the entire circumference of the person's wrist and/or forearm and a proximal portion of the band (or strap) which is proximal to the circumferential line spans between 20% and 40% of the circumference of the person's wrist and/or forearm. In an example, a first display can be larger than a second display.

In an example, in a first (non-coplanar) configuration, a first display can be located at a first radial and/or polar-coordinate location on the circumference of a person's wrist and/or forearm and a second display can be located at a second radial and/or polar-coordinate location on this circumference, wherein the first and second radial and/or polar-coordinate locations differ by between 40 and 80 degrees. In an example, in their first (non-coplanar) configuration, a first display can be in a first plane in 3D space and a second display can be in a second plane in 3D space, wherein the first plane and the second plane intersect each other in 3D space at an angle in the range of 40 to 60 degrees. In an example, the second display can be between the first display and the surface of a person's wrist and/or forearm in the first (non-coplanar) configuration.

In an example, a device can further comprise band (or strap) which attaches the first and second displays to a person's wrist and/or forearm, wherein the band (or strap) is (proximally-vs-distally) asymmetric with respect to a circumferential line around the person's wrist and/or forearm, wherein a distal portion of the band (or strap) which is distal to the circumferential line spans the entire circumference of the person's wrist and/or forearm and a proximal portion of the band (or strap) which is proximal to the circumferential line spans between 10% and 30% of the circumference of the person's wrist and/or forearm. In an example, a first display can be dorsal (e.g. closer to the most dorsal point of a circumference of a person's wrist and/or forearm) relative to a second display in a second (coplanar) configuration.

In an example, in a first (non-coplanar) configuration, a first display can be located at a first radial and/or polar-coordinate location on the circumference of a person's wrist and/or forearm and a second display can be located at a second radial and/or polar-coordinate location on this circumference, wherein the first and second radial and/or polar-coordinate locations differ by between 20 and 50 degrees. In an example, in their first (non-coplanar) configuration, a first display can be in a first plane in 3D space and a second display can be in a second plane in 3D space, wherein the first plane and the second plane intersect each other in 3D space at an angle in the range of 30 to 50 degrees. In an example, the first display can be between the second display and the surface of a person's wrist and/or forearm in the first (non-coplanar) configuration.

In an example, a device can further comprise a band (or strap), wherein the band (or strap) is connected to two sides of the first display. In an example, a display can have a cross-sectional shape which is a rounded quadrilateral. In an example, a first display can show a first portion and a second display can show a second portion of the same (image) content in a second configuration, wherein the size (e.g. area) of second portion is between 60% and 80% of the size (e.g. area) of the first portion. In an example, in a first configuration, the centroids of a first display and of a second display can be aligned along the same proximal-to-distal vector on the dorsal side (e.g. dorsal quadrant) a person's wrist and/or forearm, where proximal means closer to the person's elbow and distal means farther from the person's elbow.

In an example, in their first (non-coplanar) configuration, first and second displays can overlap. In an example, a device can further comprise a band (or strap), wherein the band (or strap) is connected to two sides of the second display. In an example, a display can have a rounded convex (e.g. circular, oval, or ellipsoidal) shape. In an example, a first display is on the dorsal side (e.g. dorsal quadrant) of a person's wrist and/or forearm, a second display is not on the dorsal side (e.g. dorsal quadrant) of the person's wrist and/or forearm in the first (non-coplanar) configuration, and the second display is on the dorsal side (e.g. dorsal quadrant) of the person's wrist and/or forearm in the second (coplanar) configuration.

In an example, in a second (coplanar) configuration, a first display can be located at a first radial and/or polar-coordinate location on the circumference of a person's wrist and/or forearm and a second display can be located at a second radial and/or polar-coordinate location on this circumference, wherein the first and second radial and/or polar-coordinate locations differ by between 10 and 30 degrees. In an example, in their first (non-coplanar) configuration, first and second displays can be adjacent. In an example, a coplanar configuration for the first and second displays means that they (e.g. their surfaces) are both in the same plane in 3D space.

In an example, a device can further comprise band (or strap) which attaches the first and second displays to a person's wrist and/or forearm, wherein the band (or strap) is (proximally-vs-distally) asymmetric with respect to a circumferential line around the person's wrist and/or forearm, wherein a distal portion of the band (or strap) which is distal to the circumferential line spans the entire circumference of the person's wrist and/or forearm and a proximal portion of the band (or strap) which is proximal to the circumferential line spans between 30% and 60% of the circumference of the person's wrist and/or forearm. In an example, a first display can be proximal (e.g. closer to a person's elbow) relative to a second display.

In an example, in a first (non-coplanar) configuration, a first display can be located at a first radial and/or polar-coordinate location on the circumference of a person's wrist and/or forearm and a second display can be located at a second radial and/or polar-coordinate location on this circumference, wherein the first and second radial and/or polar-coordinate locations differ by between 60 and 100 degrees. In an example, in their first (non-coplanar) configuration, a first display can be in a first plane in 3D space and a second display can be in a second plane in 3D space, wherein the first plane and the second plane intersect each other in 3D space at an angle in the range of 60 to 90 degrees. In an example: a first display can be on the dorsal side of a wrist and/or forearm and a second display can be on the ventral side of the wrist and/or forearm in the first configuration; and the first display can be on the dorsal side of the wrist and/or forearm and the second display can be on the dorsal side of the wrist and/or forearm in the second configuration.

In an example, a device can further comprise a band (or strap), wherein a distal side (e.g. edge) of the band (or strap) is circular or elliptical and a proximal side (e.g. edge) of the band (or strap) is undulating and/or sinusoidal. In an example, a device can further comprise a band (or strap) around a person's wrist and/or forearm, wherein the band (or strap) is connected to opposite sides of a first display, and wherein the band (or strap) is connected to a second display by arcuate elastic components (e.g. strips). In an example, a first display can have a first side (or edge) and a second side (or edge), wherein the second side is opposite the first side, a second display can have a third side (or edge) and a fourth side (or edge), wherein the fourth side is opposite the third side, wherein the first side is closer to the third side (than to the fourth side) in the first configuration, and wherein the first side is closer to the fourth side (than to the third side) in the second configuration.

In an example, in a first configuration, a first display is visible to a person but a second display is not visible to the person. In an example, in their first (non-coplanar) configuration, a first display can be in a first plane in 3D space and a second display can be in a second plane in 3D space, wherein the first plane and the second plane intersect each other in 3D space at a 40 degree angle. In an example, a device can further comprise a flexible strip around which a second display is manually rotated and/or pivoted between a first (non-coplanar) configuration and a second (coplanar) configuration. In an example, a first display and a second display can be changed from their first (non-coplanar) configuration to their second (coplanar) configuration by moving only the second display.

In an example, a first display is on the dorsal side (e.g. dorsal quadrant) of a person's wrist and/or forearm, a second display is on between the dorsal and ventral sides (e.g. dorsal and ventral quadrants) of the person's wrist and/or forearm in the first (non-coplanar) configuration, and the second display is on the dorsal side (e.g. dorsal quadrant) of the person's wrist and/or forearm in the second (coplanar) configuration. In an example, in a second (coplanar) configuration, a first display can be located at a first radial and/or polar-coordinate location on the circumference of a person's wrist and/or forearm and a second display can be located at a second radial and/or polar-coordinate location on this circumference, wherein the first and second radial and/or polar-coordinate locations differ by between 40 and 80 degrees.

In an example, the center of a first display and the center of a second display can be separated by a first distance in their first (non-coplanar) configuration and by a second distance in their second (coplanar) configuration, wherein the second distance is greater than the first distance. In an example, a device can further comprise a band (or strap), wherein a proximal side (e.g. edge) of the band (or strap) is circular or elliptical and a distal side (e.g. edge) of the band (or strap) is undulating and/or sinusoidal. In an example, a display can be a computer screen. In an example, a first display can have a first side (or edge) and a second side (or edge), wherein the second side is opposite the first side, a second display can have a third side (or edge) and a fourth side (or edge), wherein the fourth side is opposite the third side, wherein the first side is parallel to the third side in the first configuration, and wherein the first side is parallel to the fourth side in the second configuration.

In an example, in a first configuration, a second display is visible to a person but a first display is not visible to the person. In an example, in their first (non-coplanar) configuration, a first display can be in a first plane in 3D space and a second display can be in a second plane in 3D space, wherein the first plane and the second plane intersect each other in 3D space at a 90 degree angle. In an example, a device can further comprise a band (or strap), wherein the band (or strap) is connected to between 40% and 60% of the perimeter of the second display. In an example, a display can be a touch screen.

In an example, a first display can show a first portion and a second display can show a second portion of the same (image) content in a second configuration, wherein the second portion is the same size as the first portion. In an example, in a first configuration, the centers of a first display and of a second display can be located on different circumferential lines around a person's wrist and/or forearm. In an example, in their first (non-coplanar) configuration, a side (or edge) of a first display can be adjacent to a side (or edge) of a second display.

In an example, a device can further comprise band (or strap) which attaches the first and second displays to a person's wrist and/or forearm, wherein the band (or strap) is (proximally-vs-distally) asymmetric with respect to a circumferential line around the person's wrist and/or forearm, wherein a distal portion of the band (or strap) which is distal to the circumferential line spans the entire circumference of the person's wrist and/or forearm and a proximal portion of the band (or strap) which is proximal to the circumferential line does not span the entire circumference of the person's wrist and/or forearm. In an example, a first display can be dorsal (e.g. closer to the most dorsal point of a circumference of a person's wrist and/or forearm) relative to a second display.

In an example, first and second planes can be both coplanar and contiguous in the second configuration. In an example, in a second configuration, the centroids of a first display and of a second display can be aligned along the same proximal-to-distal vector on the dorsal side (e.g. dorsal quadrant) a person's wrist and/or forearm, where proximal means closer to the person's elbow and distal means farther from the person's elbow. In an example, the display surfaces of the first and second displays can be coplanar in their second configuration. In an example, a device can comprise three displays, wherein a first display is visible in a first configuration, and wherein second and third displays are visible in a second configuration.

In an example, a device can further comprise one or more additional components selected from the group consisting of: data processor; wireless data transmitter and/or receiver; and battery or other energy source. In an example, a first display can be proximal (e.g. closer to a person's elbow) relative to a second display in a first (non-coplanar) configuration. In an example, in a first configuration, a first display and a second display can both be located on the same circumferential line around a person's wrist and/or forearm. In an example, in their first (non-coplanar) configuration, a first display can be in a first plane in 3D space and a second display can be in a second plane in 3D space, wherein the first plane and the second plane intersect each other in 3D space at a 30 degree angle.

In an example: a first display can be on the dorsal side of a wrist and/or forearm and a second display can be between the dorsal and ventral sides of the wrist and/or forearm in the first configuration; and the first display can be on the dorsal side of the wrist and/or forearm and the second display can be on the dorsal side of the wrist and/or forearm in the second configuration. In an example, a device can further comprise an axle around which a second display is manually rotated and/or pivoted between a first (non-coplanar) configuration and a second (coplanar) configuration. In an example, a first display and a second display can have adjacent sides (or edges) in the second (coplanar) configuration. In an example, first and second planes can be both coplanar and adjacent in the second configuration. In an example, in a second configuration, the centers of a first display and of a second display can be located on different circumferential lines around a person's wrist and/or forearm.

In an example, the device can further comprise a band (or strap) which attaches the first display and the second display to a person's wrist and/or forearm, wherein the second display is connected to the band (or strap) by two generally-triangular members (e.g. strips). In an example, a device can further comprise a band (or strap), wherein the band (or strap) is connected to between 40% and 60% of the perimeter of the first display. In an example, a display can be a digital screen. In an example, a first display can show a first portion and a second display can show a second portion of the same (image) content in a second configuration. In an example, in a first configuration, the centers of a first display and of a second display can both be located on the same circumferential line around a person's wrist and/or forearm.

In an example, in their first (non-coplanar) configuration, a side (or edge) of a first display can be parallel to a side (or edge) of a second display. In an example, a device can further comprise an axle around which a second display rotates and/or pivots between a first (non-coplanar) configuration and a second (coplanar) configuration. In an example, a first display and a second display can display different sections of the same (image) content. In an example, coplanar means being in the same plane in 3D space. In an example, in a second configuration, the centers of a first display and of a second display can both be located on the same circumferential line around a person's wrist and/or forearm. In an example, the device can further comprise a band (or strap) which attaches the first display and the second display to a person's wrist and/or forearm, wherein the second display is connected to the band (or strap) by two arcuate (e.g. convex) strips.

With the conclusion of the introductory section which covered general concepts, components, and methods for this invention, this disclosure now discusses the specific examples shown in FIGS. 1 through 4.

FIGS. 1 and 2 show two sequential views of a forearm-wearable computing device with a large display area comprising: (a) primary attachment member 101, wherein this primary attachment member is configured to attach the device to a person's wrist and/or forearm by encircling at least 50% of the circumference of the person's wrist and/or forearm; (b) folding attachment member 102, wherein this folding attachment member has a first configuration in which it overlaps the primary attachment member by a first amount, wherein this folding attachment member has a second configuration in which it overlaps primary attachment member by a second amount, wherein the second amount is less than the first amount, and wherein the folding attachment member is moved from the first configuration to the second configuration by rotation of the folding attachment member relative to the primary attachment member; (c) first display member 103, wherein this first display member is on the exterior surface of the device and visible to the person when the folding attachment member is in the first configuration; (d) second display member 104, wherein this second display member is on the exterior surface of the device and visible to the person when the folding attachment member is in the second configuration; and (e) third display member 105, wherein this third display member is on the exterior surface of the device and visible to the person when the folding attachment member is in the second configuration.

FIG. 1 shows this device when folding attachment member 102 is in the first configuration. FIG. 2 shows this device when folding attachment member 102 has been moved into the second configuration. This movement is symbolically represented by a dotted-line arrow in FIG. 2. In FIG. 2, folding attachment member 102 has been moved from the first configuration to the second configuration by being rotated around a side-to-side axial joint which connects the folding attachment member to the primary attachment member. In this example, the axial joint is along the surface of the person's wrist and/or forearm and the edge of the folding attachment member which is opposite to the axial joint is rotated away from the surface of the wrist and/or forearm during the transition from the first configuration to the second configuration. In FIG. 2, folding attachment member 102 has been moved manually (e.g. by the person) from the first configuration to the second configuration. In another example, a folding attachment can be automatically moved from the first configuration to the second configuration by an actuator which is part of the device.

In the example shown in FIG. 2, the device can be said to have been “flipped open” when folding attachment member 102 has been moved from the first configuration to the second configuration. In this example, folding attachment member 102 completely overlaps primary attachment member 101 in the first configuration and does not overlap primary attachment member 101 at all in the second configuration. In this example, the folding attachment member lies flat against the surface of the person's wrist and/or forearm in the second configuration. In this example, folding attachment member 102 “flips open” in a proximal direction—moving closer to the person's elbow in the transition from the first configuration to the second configuration. In another example, folding attachment member 102 could “flip open” in a distal direction—moving away from the person's elbow in the transition from the first configuration to the second configuration.

In an example, primary attachment member 101 can be a band, strap, chain, bracelet, or bangle. In an example, primary attachment member 101 can be a flexible and/or elastic band, strap, mesh, cuff, or chain which spans the entire circumference of the person's wrist and/or forearm. In an example, primary attachment member 101 can be sufficiently flexible and/or elastic that it can be slipped over the hand onto the wrist and/or forearm. In an example, primary attachment member 101 can be a band, strap, or chain which further comprises a buckle, clasp, clip, hook, hook-and-eye material, pin, latch, button, and/or zipper which fastens it around the circumference of the person's wrist and/or forearm. In an example, primary attachment member 101 can be a bracelet or bangle. In an example, primary attachment member 101 can be sufficiently rigid and/or resiliently-flexible that it holds the device on the person's wrist and/or forearm even though it does not span the entire circumference of the person's wrist and/or forearm.

In an example, folding attachment member 102 can be a band, strap, or protrusion. In an example, folding attachment member 102 can be a flexible and/or elastic band, strap, or protrusion which spans less of the circumference of the person's wrist and/or forearm than is spanned by primary attachment member 101. In an example, folding attachment member 102 may span only the upper (or frontal) surface of the person's wrist and/or arm. In an example, a folding attachment member can have arcuate edges which connect to the lateral sides of the second display member. Such arcuate edges can reduce snagging of the second display member in the second configuration.

In an example, at least 75% of folding attachment member 102 overlaps primary attachment member 101 in the first configuration. In an example, at least 90% of folding attachment member 102 overlaps primary attachment member 101 in the first configuration. In an example, folding attachment member 102 completely overlaps primary attachment member 101 in the first configuration. In an example, less than 75% of folding attachment member 102 overlaps primary attachment member 101 in the second configuration. In an example, less than 10% of folding attachment member 102 overlaps primary attachment member 101 in the second configuration. In an example, folding attachment member 102 does not overlap primary attachment member 101 at all in the second configuration.

In an example, a folding attachment member can be connected to a primary attachment member by a side-to-side (lateral) axial joint. In an example, the surface area of a person's wrist or arm which is covered by the device can be increased when the folding attachment member is moved from the first configuration to the second configuration. This enables the outward-facing surface device to be smaller when only the first display member is in use and larger when the second and third display members are in use. In an example the folding attachment member can be parallel to the plane of the primary attachment member in the first configuration. In an example the folding attachment member can be parallel to the plane of the primary attachment member in both the first configuration and second configuration. In an example, the folding attachment member may temporarily not be parallel to the plane of the primary attachment member during the transition from the first configuration to the second configuration.

In an example, a display member can be a computer display screen. In an example, a display member can have a cross-sectional shape which is selected from the group consisting of: rectangular with rounded vertexes, hexagonal with rounded vertexes, square, rectangular, hexagonal, circular, elliptical, and oblong. In an example, the second and third display members can be centrally aligned along the same proximal-to-distal axis of the person's wrist and/or forearm. In an example, a display member can have a flat display surface. In an example, a display member can be a touch screen which responds to finger movements. In this example, the first, second, and third display members are separate components. In another example, the first and third display members can be top and bottom views of the same component. In another example, the device may not have a first display member and only have the second and third display members.

In an example, the first display member can display a summary of the information which is displayed in more detail on the second and third display members. In an example, the second and third display members can display two different sections of the same text content. In an example, the second and third display members can display two different sections of the same image content. In an example, the second and third display members can display text and image, respectively, from the same multi-media content.

In an example, a display member can display information with a proximal-to-distal or distal-to-proximal orientation. In an example, a display member can display information with a lateral orientation. In an example, the device can automatically change the radial orientation of information on a display member based on the orientation and/or movement of the device as detected by a gyroscope and/or motion sensor which is incorporated into the device. In an example, the orientation of information on a display member can be automatically changed based on the orientation and/or position of the device relative to the person's eyes. In an example, the orientation and/or position of the device relative to the person's eyes can be determined based on data from one or more cameras, motion sensors, inertial sensors, and/or bend sensors which are incorporated into the device.

In an example, a forearm-wearable device can further comprise one or more sensors. In an example, a sensor can be a multi-axial accelerometer. In an example, a sensor can be a gyroscope. In an example, a sensor can be a light energy sensor. In an example, a sensor can be a spectroscopic sensor which collects data concerning the spectrum of light reflected from and/or transmitted through tissue of the person's wrist and/or forearm. In an example, a sensor can be an electromagnetic energy sensor. In an example, a sensor can measure the resistance, impedance, and/or conductivity of tissue of the person's wrist and/or forearm with respect to the transmission of electromagnetic energy. In an example, a sensor can measure electromagnetic energy emitted from muscles and/or nerves in the person's wrist and/or forearm. In an example, a sensor can be a capacitive electromagnetic energy sensor.

In an example, a sensor can be an environmental light energy sensor. In an example, a display member can have a first display mode which requires less energy and a second display mode which requires more energy. In an example, a display member can automatically switch from the first display mode to the second display mode when the second display mode is required for the person to be able to see information on the display member. In an example, the device can switch the display member from the first mode to the second mode when there is a high level of environmental light energy and the display member would not be visible in bright light in the first display mode. In an example, the device can switch the display member from the first mode to the second mode when there is a low level of environmental light energy and the display member would not be visible in dim light (or darkness) in the first display mode.

In an example, this device can change one or more display members from a first (lower energy) display mode to a second (higher energy) display mode, or vice versa, based on data from one or more motion and/or inertial sensors which are incorporated into the device or with which the device is in wireless communication. In an example, when a motion and/or inertial sensor indicates a first level of movement of the device or a first orientation of the device, then the assumption is that the person is not looking at the device and the device sets the display member to the first (lower energy) display mode. In an example, when a motion and/or inertial sensor indicates a second level of movement of the device or a second orientation of the device, then the assumption is that the person may be looking at the device and the device sets the display member to the second (higher energy) display mode.

In an example, this device can be part of a multi-device system which includes other locations on a person's body, such as the person's head or torso. In an example, this device can be in communication with a motion and/or inertial sensor which is located elsewhere on the person's body so that the relative motion or relative orientation of the device (relative to the rest of the person's body) can be monitored. Measuring the relative motion or orientation of the device (e.g. relative to the rest of the person's body) rather than absolute motion or position of the device (e.g. relative to the earth) can help to factor out changes in motion or orientation which are due to being in a car, elevator, or airplane. Even though the absolute position or orientation of a device might be changing rapidly in a car, elevator, or airplane, the relative position of the device (relative to the rest of the person) may be stable and the person may wish see the display. Example variations discussed elsewhere in this disclosure or in priority-linked disclosures can also be applied to this example where relevant.

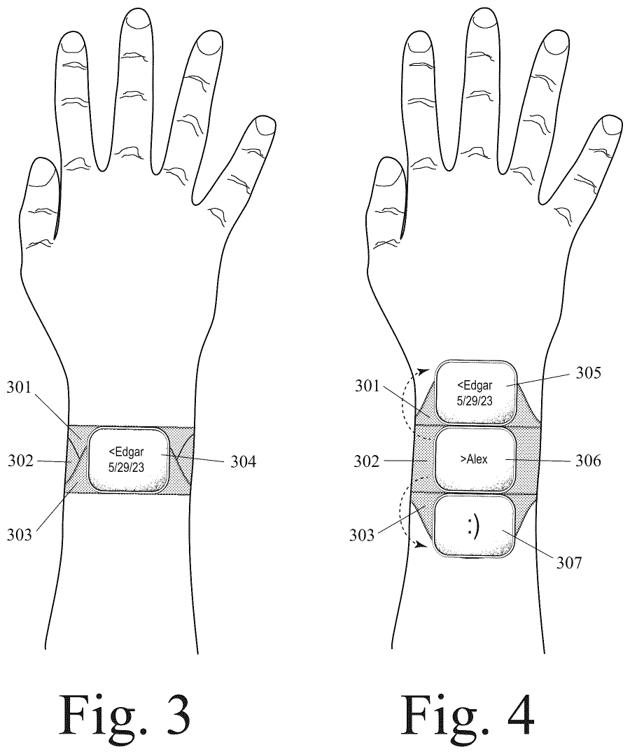

FIGS. 3 and 4 show two sequential views of a forearm-wearable computing device with a large display area comprising: (a) primary attachment member 302, wherein this primary attachment member is configured to attach the device to a person's wrist and/or forearm by encircling at least 50% of the circumference of the person's wrist and/or forearm; (b) first folding attachment member 303, wherein this first folding attachment member has a first configuration in which it overlaps the primary attachment member by a first amount, wherein this first folding attachment member has a second configuration in which it overlaps primary attachment member by a second amount, wherein the second amount is less than the first amount, and wherein this first folding attachment member is moved from the first configuration to the second configuration by rotation relative to the primary attachment member; (c) second folding attachment member 303, wherein this second folding attachment member has a third configuration in which it overlaps the primary attachment member by a third amount, wherein this second folding attachment member has a fourth configuration in which it overlaps the primary attachment member by a fourth amount, wherein the fourth amount is less than the third amount, and wherein this second folding attachment member is moved from the third configuration to the fourth configuration by rotation relative to the primary attachment member; (d) first display member 304, wherein this first display member is on the exterior surface of the device and visible to the person when the first folding attachment member is in the first configuration; (e) second display member 307, wherein this second display member is on the exterior surface of the device and visible to the person when the first folding attachment member is in the second configuration; (f) third display member 305, wherein this third display member is on the exterior surface of the device and visible to the person when the second folding attachment member is in the fourth configuration; and (g) fourth display member 306, wherein this fourth display member is on the exterior surface of the device and visible to the person when the first folding attachment member is in the second configuration and the second folding attachment member is in the fourth configuration. Example variations discussed elsewhere in this disclosure or in priority-linked disclosures can also be applied to this example where relevant.

I claim:

1. A wearable computing device comprising:
a primary attachment member, wherein this primary attachment member is configured to attach the device to a person's wrist and/or forearm by encircling at least 50% of the circumference of the person's wrist and/or forearm;
a folding attachment member, wherein this folding attachment member has a first configuration in which it overlaps the primary attachment member by a first amount, wherein this folding attachment member has a second configuration in which it overlaps primary attachment member by a second amount, wherein the second amount is less than the first amount, and wherein the folding attachment member is moved from the first configuration to the second configuration by rotation of the folding attachment member relative to the primary attachment member;
a first display member, wherein this first display member is on the exterior surface of the device and visible to the person when the folding attachment member is in the first configuration; and
a second display member, wherein this second display member is on the exterior surface of the device and visible to the person when the folding attachment member is in the second configuration.

2. The device in claim 1 wherein the device further comprises a third display member, wherein this third display member is on the exterior surface of the device and visible to the person when the folding attachment member is in the second configuration.

3. The device in claim 1 wherein the primary attachment member is a band, strap, chain, bracelet, or bangle.

4. The device in claim 1 wherein the folding attachment member has arcuate edges which connect to the lateral sides of the second display member.

5. The device in claim 1 wherein at least 90% of the folding attachment member overlaps the primary attachment member in the first configuration.

6. The device in claim 1 wherein less than 10% of the folding attachment member overlaps primary attachment member in the second configuration.

7. The device in claim 1 wherein a display member is a computer display screen.

8. A wearable computing device comprising:
a primary attachment member, wherein this primary attachment member is configured to attach the device to a person's wrist and/or forearm by encircling at least 50% of the circumference of the person's wrist and/or forearm;
a folding attachment member, wherein this folding attachment member has a first configuration in which it overlaps the primary attachment member by a first amount, wherein this folding attachment member has a second configuration in which it overlaps primary attachment member by a second amount, wherein the second amount is less than the first amount, and wherein the folding attachment member is moved from the first configuration to the second configuration by rotation of the folding attachment member relative to the primary attachment member;
a first display member, wherein this first display member is on the exterior surface of the device and visible to the person when the folding attachment member is in the first configuration;
a second display member, wherein this second display member is on the exterior surface of the device and visible to the person when the folding attachment member is in the second configuration; and;
a third display member, wherein this third display member is on the exterior surface of the device and visible to the person when the folding attachment member is in the second configuration.

9. The device in claim 8 wherein the primary attachment member is a band, strap, chain, bracelet, or bangle.

10. The device in claim 8 wherein the folding attachment member has arcuate edges which connect to the lateral sides of the second display member.

11. The device in claim 8 wherein at least 90% of the folding attachment member overlaps the primary attachment member in the first configuration.

12. The device in claim 8 wherein less than 10% of the folding attachment member overlaps the primary attachment member in the second configuration.

13. The device in claim 8 wherein a display member is a computer display screen.

14. A wearable computing device comprising:
a first display which is attached to a person's wrist and/or forearm; and
a second display which is attached to the person's wrist and/or forearm, wherein the first display and the second display have a first configuration in which the first display and the second display are not coplanar, wherein the first display and the second display have a second configuration in which the first display and the second display are coplanar.

15. The device in claim 14 wherein the device further comprises a band or strap which attaches the first display and the second display to the person's wrist and/or forearm.

16. The device in claim 14 wherein the first display and the second display are moved from their first configuration to their second configuration by rotating and/or pivoting the second display relative to the first display.

17. The device in claim 14 wherein the first display and the second display are moved from their first configuration to their second configuration by sliding and/or telescoping the second display relative to the first display.

18. The device in claim 14 wherein the first display and the second display are moved from their first configuration to their second configuration by flipping and/or unfolding the second display relative to the first display.

19. The device in claim 14 wherein the first display is on the dorsal quadrant of the person's wrist and/or forearm, the second display is on the ventral quadrant of the person's wrist and/or forearm in the first configuration, and the second display is on the dorsal quadrant of the person's wrist and/or forearm in the second configuration.

20. The device in claim 14 wherein the first display is on the dorsal quadrant of the person's wrist and/or forearm, the second display is between the dorsal and ventral quadrants of the person's wrist and/or forearm in the first configuration, and the second display is on the dorsal quadrant of the person's wrist and/or forearm in the second configuration.
